(19) United States
(12) Patent Application Publication
(10) Pub. No.: US 2003/0060780 A1
(43) Pub. Date: Mar. 27, 2003

(54) Epidermic injection device

(76) Inventor: Hang Chung Shu, Taipei (TW)

Correspondence Address: RABIN & CHAMPAGNE, P.C., Suite 500, 1101 14th Street, N.W., Washington, DC, 20005, US

(21) Appl. No.: 09/960,556

(22) Filed: Sep. 24, 2001

(51) Int. Cl. A61M 5/00

(52) U.S. Cl. 604/257

(57) ABSTRACT

An epidermic injection device includes a flexible and elastic storing chamber. The storing chamber includes a flat contact surface for contacting with a user's skin. A plurality of thin needles are distributed over the contact surface with their sharp tips outward and perpendicularly projected from the contact surface. Each needle has an internal passage communicating with a hollow space in the storing chamber. Liquid medicine is first injected into the hollow space to generate an internal pressure in the storing chamber. When the device is attached at the flat contact surface to the user's skin or mucous membrane, the needles pierce through the skin and the internal pressure urges the liquid medicine to automatically slowly flow through the needles into the skin. The device largely reduces sharp pains that would be otherwise caused by a conventional syringe during injection, and is particularly suitable for use in epidermic local anesthesia.

### FIELD OF THE INVENTION

The present invention relates to an epidermic injection device, and more particularly to an automatic injection device that does not use any long cannula to pierce through a user's skin and therefore largely reduces pains possibly caused in the injection.

### BACKGROUND OF THE INVENTION

In a conventional injection of an amount of liquid medicine into a patient's body, a sharp cannula on a syringe is pierced through the patient's skin, and a plunger is pushed to urge the liquid medicine in a barrel of the syringe through the cannula into the skin. When the cannula pierces through the skin, it would normally cause a sharp pain to frighten most patients.

For example, a dental surgery in the oral cavity usually requires a local anesthesia at the gum. When a pointed cannula is pierced through the gum to inject an amount of anesthetic, the patient would feel a sharp pain. In the case the patient is a child, such injection of anesthetic is particular difficult to complete.

### SUMMARY OF THE INVENTION

It is therefore a primary object of the present invention to provide an epidermic injection device to improve the conventional syringe by reducing the pains possibly caused in the injection.

To achieve the above and other objects, the injection device of the present invention mainly includes a storing chamber made of a flexible and elastic material. The storing chamber includes a flat contact surface for contacting with a user's skin. A plurality of thin needles are distributed over the contact surface with their sharp tips outward and perpendicularly projected from the contact surface. Each needle has an internal passage communicating with a hollow space in the storing chamber. Liquid medicine is first injected into the hollow space to generate an internal pressure in the storing chamber. When the device is attached at the flat contact surface to the user's skin or mucous membrane, the sharp tips of the needles easily pierce through the skin and the internal pressure urges the liquid medicine to automatically slowly flow through the needles into the skin. Alternatively, an external pressure may be applied on the storing chamber to compress the liquid medicine through the needles. The device largely reduces the sharp pains that would be otherwise caused by a conventional syringe during injection, and is particularly suitable for use in epidermic local anesthesia.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a perspective view of an epidermic injection device according to the present invention;

FIG. 2 is a sectional view of the epidermic injection device of FIG. 1; and

FIGS. 3A to 3C illustrate the use of the epidermic injection device of the present invention.

### DETAILED DESCRIPTION OF THE PREFERRED EMBODIMENTS

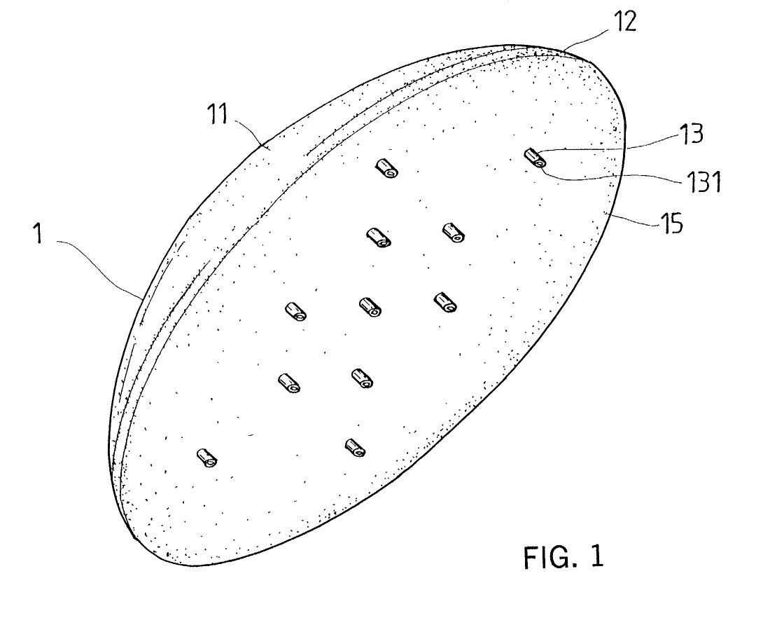

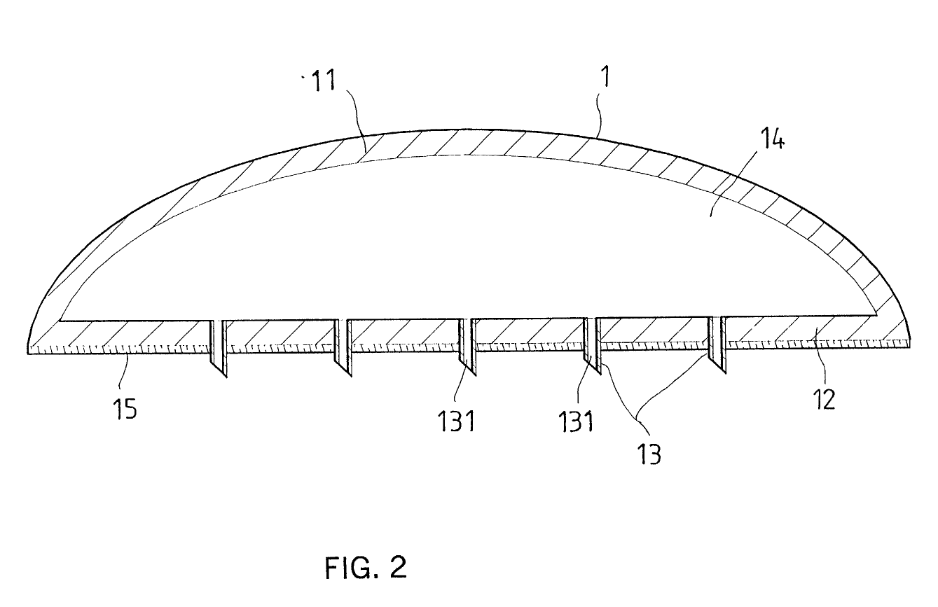

Please refer to FIGS. 1 and 2 that are perspective and sectional views, respectively, of an epidermic injection device of the present invention. As shown, the epidermic injection device mainly includes a storing chamber 1 made of a flexible and elastic material, such as rubber or silicon rubber material. The storing chamber 1 includes a top 11 that is preferably a curved surface, and a bottom 12 that is a flat contact surface. The top 11 and the bottom 12 together define a hollow space 14 between them for storing liquid medicine for injection. The bottom 12 is a surface suitable for contacting with an outer surface of a user's skin. A layer of glue backing 15 may be provided on an outer surface of the bottom 12 for adhesively attaching to the user's skin or mucous membrane at where the liquid medicine is to be injected. A plurality of very thin and short needles 13 are distributed over the bottom 12 to extend in a direction perpendicular to the bottom 12. Each needle 13 has a sharp tip outward projected from the bottom 12 to enable easy piercing of the needle 13 through an epidermis. Each needle 13 defines an internal passage 131 communicating with the hollow space 14 in the storing chamber 1, so that liquid medicine stored in the storing chamber 1 could flow through the passages 131 of the needles 13 when the storing chamber 1 is compressed.

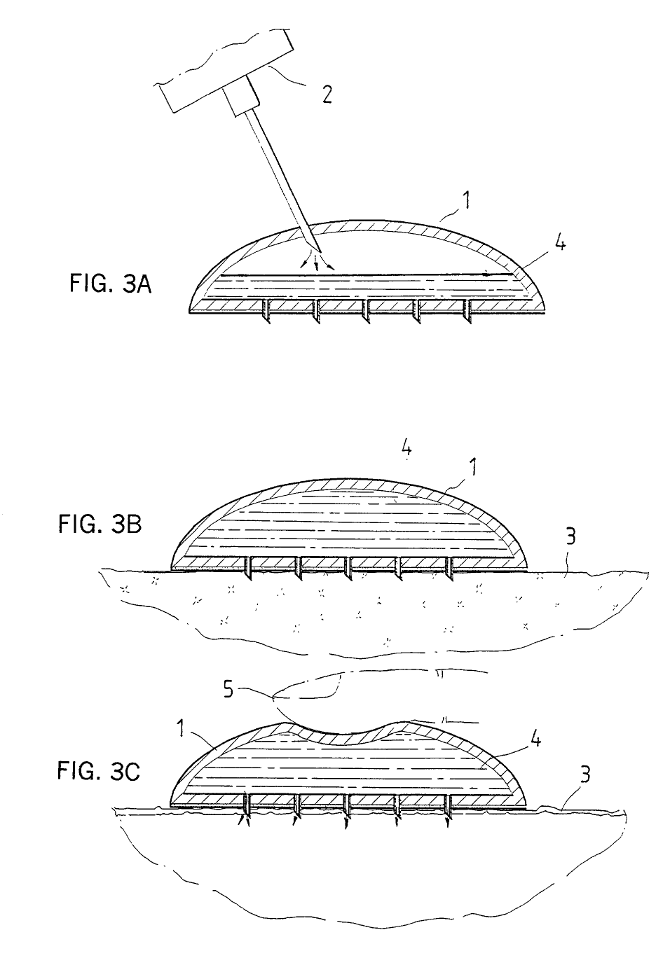

Please refer to FIGS. 3A to 3C that illustrate the use of the epidermic injection device of the present invention. First, use a general syringe 2 to draw up an adequate amount of liquid medicine 4 and pierce a cannula of the syringe 2 through the top 11 of the storing chamber 1 to fill the hollow space 14 with the liquid medicine 4, as shown in FIG. 3A. The syringe 2 is then extracted from the storing chamber 1. Since the storing chamber 1 is made of a flexible and elastic material, piercing and extracting of the syringe 2 through and from the top 11 would not produce any hole on the storing chamber 1 to cause any leakage of the liquid medicine 4. Thereafter, attach the bottom 12 to an outer surface of a user's skin 3, as shown in FIG. 3B. At this point, the needles 13 pierce through the skin 3, and injection of the liquid medicine 4 starts. Since the storing chamber 1 is made of a flexible and elastic material, the liquid medicine 4 injected into the storing chamber 1 generates an internal pressure in the storing chamber 1. The internal pressure enables the liquid medicine 4 to automatically slowly flow from the hollow space 14 through the passages 131 of the needles 13 into the user's skin. Alternatively, as shown in FIG. 3C, the curved top 11 may be lightly depressed with a finger 5 to apply a pressure to the storing chamber 1 and deform the latter, so that the liquid medicine 4 in the hollow space 14 is urged through the passages 131 of the needles 13 into the user's skin 3.

Unlike the conventional injection with a syringe that would usually result in sharp pains, the epidermic injection device of the present invention causes only a very minor sting and numbness when it is attached to the skin 3, because the needles 13 have a very short length. The present invention therefore largely reduces the user's fear and enables easy injection of liquid medicine 4.

The epidermic injection device of the present invention may be employed, for example, in tooth extraction or nerve removal in dental surgery to inject an anesthetic. Since the device does not cause too much pain, it largely reduces difficulties in injection. In particular, the liquid medicine 4 automatically slowly flows from the storing chamber 1 into the user's skin 3 with or without a minor depression of the top 11 so as to complete the injection. The device is particularly suitable for use when the user is a child. The device does not cause any pain to make the child cry and resist, allowing the injection to be smoothly completed.

Further, the liquid medicine 4 injected into the hollow space 14 generates a pressure inside the storing chamber 1, just like the principle of inflating a balloon. This internal pressure enables the liquid medicine 4 to automatically and slowly flow from the hollow space 14 through the thin needles 13 into the user's skin 3. The user almost feels no pain during the injection.

With the above arrangements, the epidermic injection device of the present invention is novel and improved because it largely reduces the pains that would be otherwise caused by the conventional syringe injection.

What is claimed is:

1. An injection device for injecting a liquid medicine into a user's skin, comprising:
a storing chamber made of a flexible and elastic material to define a hollow space therein for storing an amount of liquid medicine, said storing chamber including at least a flat contact surface for attaching to and contacting with the user's skin; and
a plurality of short and thin needles distributed over said flat contact surface of said storing chamber, each said needle defining an internal passage communicating with said hollow space of said storing chamber, and having a sharp tip outward projected from said flat contact surface, so that said sharp tip of each said needle pierces through the user's skin when said flat contact surface is attached to the user's skin; and
said liquid medicine being injected into said hollow space of said storing chamber to generate an internal pressure inside said storing chamber, and
said liquid medicine automatically and slowly flowing from said storing chamber through said passages of said needles into the user's skin due to said internal pressure, or due to an external pressure applied to deform said storing chamber.

2. The injection device as claimed in claim 1, wherein said storing chamber includes a curved top above said flat contact surface, so that said curved top and said flat contact surface together define said hollow space between them.

3. The injection device as claimed in claim 1, wherein said flat contact surface of said storing chamber is provided at an outer surface with a layer of glue backing.
